(19) United States
(12) Patent Application Publication
(10) Pub. No.: US 2005/0171487 A1
(43) Pub. Date: Aug. 4, 2005

(54) Connector and tubing assembly for use with a syringe

(75) Inventors: John A. Haury, Sewickly, PA (US); Edward J. Rhinehart, Monroeville, PA (US)

Correspondence Address: GREGORY L BRADLEY;MEDRAD INC, ONE MEDRAD DRIVE, INDIANOLA, PA, 15051, US

(73) Assignee: Medrad, Inc.

(21) Appl. No.: 11/099,147

(22) Filed: Apr. 5, 2005

Related U.S. Application Data
(63) Continuation of application No. 10/140,707, filed on May 7, 2002.
Application No. 10/140,707 is a division of application No. 09/362,833, filed on Jul. 28, 1999, now abandoned.
(60) Provisional application No. 60/097,371, filed on Aug. 21, 1998.

(51) Int. Cl. A61M 25/16

(52) U.S. Cl. 604/239; 604/533; 604/240; 264/242; 264/255; 29/464

(57) ABSTRACT

An assembly and a method for connecting a syringe to an injector and a catheter includes a connector adapted to be releasably attached to the syringe. The assembly further includes tubing connected to the connector such that the tubing is in sealed fluid connection with the syringe when the connector is attached to the syringe. The tubing is connected to the connector in a manner to prevent disconnection thereof before and during connection of the connector to the syringe.

### CROSS REFERENCE TO RELATED APPLICATIONS

This application is a continuation of application Ser. No. 10/140,707, filed on May 7, 2002, which was a division of application Ser. No. 09/362,833 filed on Jul. 28, 1999, which claims the benefit of Provisional Application Ser. No. 60/097,371, filed on Aug. 21, 1998, the contents of which are hereby incorporated by reference.

### BACKGROUND OF THE INVENTION

1. Field of the Invention

The present invention relates to an assembly for use with a syringe and, more particularly, to a connector and tubing assembly suitable for use with an injector-actuated prefilled syringe.

2. Description of Related Art

A number of injector-actuated syringes and powered injectors for use in medical procedures such as angiography, computed tomography and NMR/MRI have been developed. For example, U.S. Pat. No. 4,006,736 discloses an apparatus for injecting fluid into the vascular system of a human being or an animal. Likewise, U.S. Pat. No. 4,677,980 discloses an angiographic injector and syringe including a rotating turret for housing two angiographic syringes in readiness for injection. Furthermore, U.S. Pat. No. 5,383,858 discloses a front-loading injector and a syringe mountable thereon for injection procedures.

Over the past few years, prefilled syringes have become increasingly desirable and popular for use with powered injectors. Because prefilled syringes, in contrast with more conventional empty syringes, already contain the contrast media to be injected into a patient, their use saves medical practitioners time and effort in preparing and conducting injection procedures.

A typical prefilled syringe, such as the Ultraject® prefilled syringe marketed by Mallinckrodt Medical, Inc., is packaged in a sealed tray along with a syringe nut package containing a sterile syringe nut. The inside of the prefilled syringe (i.e., the portion in contact with the contrast media) is sterile to prevent patient contamination. The exterior surfaces of the prefilled syringe and the syringe nut package are not sterile.

A sterile connecting tube is used to provide a fluid pathway between the prefilled (or conventional) syringe and a catheter in a patient. The sterile connecting tube includes male and female luer connectors at respective ends thereof and is provided in a separate package.

According to sterile handling procedures instituted to prevent patient contamination, a non-sterile technician in the injection suite must open the sealed tray containing the prefilled syringe and the syringe nut package, and load the prefilled syringe in the injector. The non-sterile technician opens the syringe nut package to provide access to the sterile syringe nut and removes the tip cap from the nozzle of the prefilled syringe. A second, sterile technician in the injection suite removes the sterile syringe nut from the opened syringe nut package, and attaches it to the prefilled syringe. The non-sterile technician opens the connecting tube package and the sterile technician removes the sterile connecting tube therefrom, and engages one luer end of the connecting tube with the syringe nut. While maintaining the luer end of the connecting tube in engagement with the syringe nut, the sterile technician connects the syringe nut to the nozzle of the prefilled syringe to create a sterile luer connection between the syringe and the connecting tube. The sterile technician then connects the other end of the connecting tube to the catheter inserted in the patient to provide a sterile luer connection therebetween. The injection procedure then may be conducted.

As can be readily perceived, the sterile procedure required to properly load and connect the above-described prefilled syringe, syringe nut and connecting tube requires various handling steps. In addition, because non-sterile surfaces are present on the syringe and the syringe nut package, the possibility of patient contamination exists, even if strict attention is paid by medical personnel to proper sterile handling procedures.

To reduce the risk of patient contamination, and to reduce the time and effort required to properly load and connect prefilled (and other) syringes and their requisite syringe nuts and connecting tubes, it is desirable to develop a syringe and tubing assembly that reduces the number of non-sterile surfaces and handling steps to prepare for an injection procedure.

### SUMMARY OF THE INVENTION

The present invention provides a syringe and tubing assembly that reduces the number of non-sterile surfaces and handling steps to prepare for an injection procedure, which minimizes the possibility of contaminating the fluid path and reduces the time required to properly load and connect a syringe to an injector. In a preferred embodiment, the present invention provides these advantages by providing a sterile combined syringe nut and connecting tubing assembly.

According to a first aspect of the present invention, a method for connecting a syringe to a catheter to inject a fluid into a patient is provided. The method includes providing a sterile syringe, preferably a prefilled syringe, and a sterile connector and tubing assembly. The connector is connected to the nozzle of the syringe to provide a sterile connection between the syringe and the tubing. The other end of the tubing is connected to a catheter inserted into a patient.

In a preferred embodiment, the syringe and the connector and tubing assembly are provided in separate sterile packages, and medical personnel are able to load the syringe and connect the connector and tubing assembly thereto and to the patient to provide a sterile fluid pathway between the syringe and the patient.

According to a second aspect of the present invention, an assembly for connecting a syringe to a catheter to inject a fluid into a patient is provided. The assembly includes generally a connector adapted to be releasably attached to the syringe, and a length of tubing connected to the connector. The tubing is connected to the connector such that the tubing is in sealed fluid connection with the syringe when the connector is attached to the syringe. The tubing is connected to the connector in a prevent disconnection thereof before and during connection of the connector to the syringe.

The connector is preferably rotatable relative to the tubing to facilitate connection with the syringe. The connector and the syringe may, for example, include cooperating threaded portions to form a releasable connection between the assembly and the syringe. In a preferred embodiment, the syringe is a prefilled syringe having a sterile interior surface, and the tubing and the syringe form a sterile luer connection upon connection of the connector to the syringe, as well known in the medical arts. Such a luer connection provides a sealed engagement even at relatively high pressures, such as those experienced in angiographic procedures.

According to a third aspect of the present invention, a method of manufacturing a connector and tubing assembly as an assembled product is provided. The method includes providing a first polymeric material having a first melting point, providing a second polymeric material having a second melting point that is less than the first melting point, molding the connector from the first material, molding an end of the tubing from the second material at substantially the same time, and cooling the first material and the second material, whereby the connector is able to rotate with respect to the end of the tubing when the first material and the second material harden.

The present invention provides a number of significant advantages over prior assemblies and methods for attaching tubing to syringes. The connector and tubing assembly of the present invention can, for example, be shipped in a single sterile package. Furthermore, by combining the syringe nut connector and the connecting tube, the preferred embodiment of the present invention eliminates the need for separate syringe nuts and their packaging, and thereby reduces the number of components that must be handled by medical personnel to prepare for an injection procedure. Because there are less components and non-sterile surfaces for medical personnel to handle, the present invention reduces the risk of patient contamination. Moreover, because the connector and the tubing are pre-connected in a preferred embodiment, it is much easier for the operator to quickly form a sealed engagement between the syringe and the catheter with the assembly of the present invention than is possible with currently available connector/tubing combinations.

The present invention, along with further aspects and attendant advantages, will best be understood by reference to the following detailed description taken in conjunction with the accompanying drawings.

### BRIEF DESCRIPTION OF THE DRAWINGS

FIG. 1 is a flow chart illustration of the current methodology for loading a syringe onto an injector for an injection procedure;

FIG. 2 is a flow chart illustration of the methodology of the present invention for loading a syringe onto an injector for an injection procedure;

FIG. 3 illustrates an embodiment of a currently available connector/tubing combination used in injection procedures;

FIG. 4A illustrates an embodiment of an assembly of the present invention;

FIG. 4B illustrates the assembly of FIG. 4A before connection thereof during manufacture;

FIG. 5A illustrates the assembly of FIG. 4A aligned with a syringe before connection thereto;

FIG. 5B illustrates the assembly of FIG. 4A in sealing engagement with the syringe; and

FIG. 5C illustrates an enlarged view of the sealing engagement of FIG. 4B.

### DESCRIPTION OF THE PREFERRED EMBODIMENTS

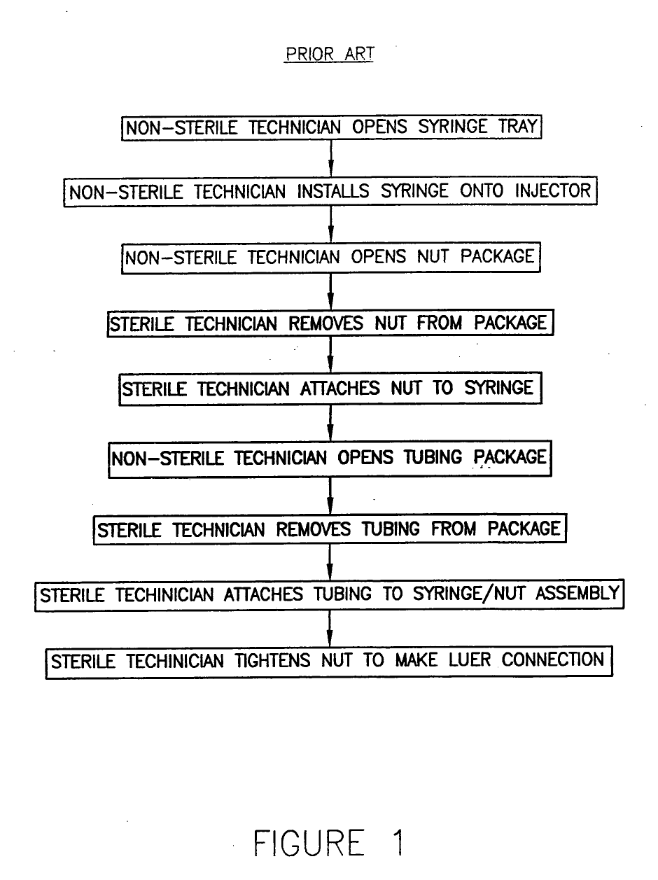

As recited above and as illustrated in FIG. 1, the conventional method for loading a prefilled syringe onto an injector includes many time-consuming steps and requires the handling of a number of components having non-sterile surfaces, thereby increasing the risk of patient contamination.

Specifically, as shown in FIG. 1, the following method is currently performed for installing or loading a syringe onto an injector: (1) a non-sterile technician in the injection suite opens the sealed tray containing a syringe and a syringe nut package; (2) the non-sterile technician installs or loads the syringe onto the injector; (3) the non-sterile technician opens the syringe nut package to provide access to the sterile syringe nut and removes the tip cap from the nozzle of the syringe; (4) a second, sterile technician in the injection suite removes the sterile syringe nut from the opened syringe nut package; (5) the sterile technician attaches the syringe nut to the syringe; (6) the non-sterile technician opens the connecting tube package; (7) the sterile technician removes the sterile connecting tube from the package; (8) the sterile technician attaches one luer end of the connecting tubing to the syringe nut; and (9) while maintaining the luer end of the connecting tube in engagement with the syringe nut, the sterile technician tightens the syringe nut on the nozzle of the syringe to make the luer connection between the syringe and the connecting tube. To complete the fluid path from the syringe to the patient, the sterile technician then connects the other end of the connecting tube to the catheter inserted in the patient to provide a sterile luer connection therebetween. As can be seen in FIG. 1, at least nine (9) steps are currently conducted to install a syringe onto an injector and to connect the connecting tubing thereto.

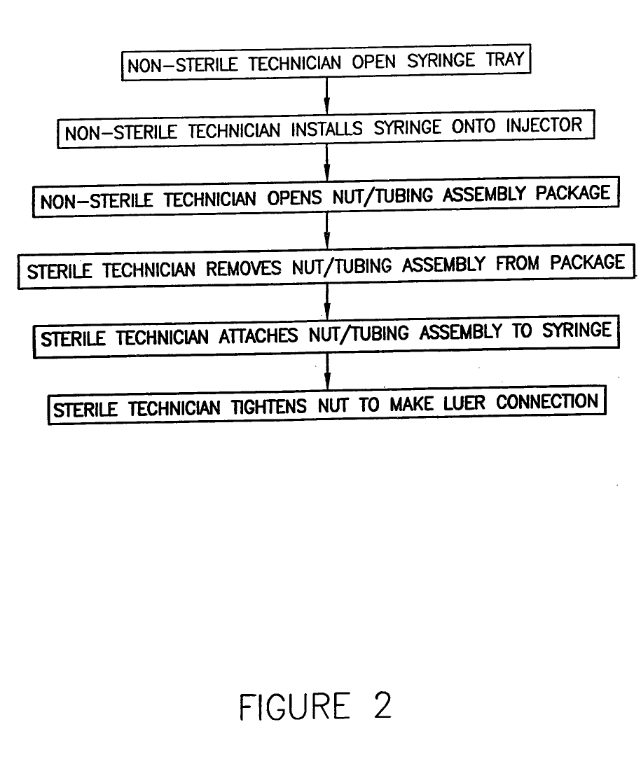

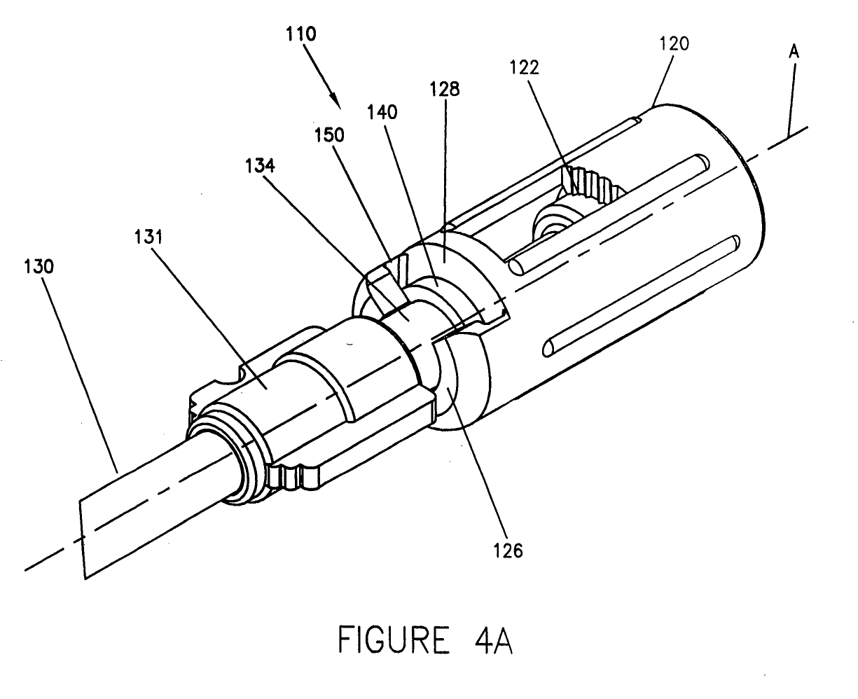

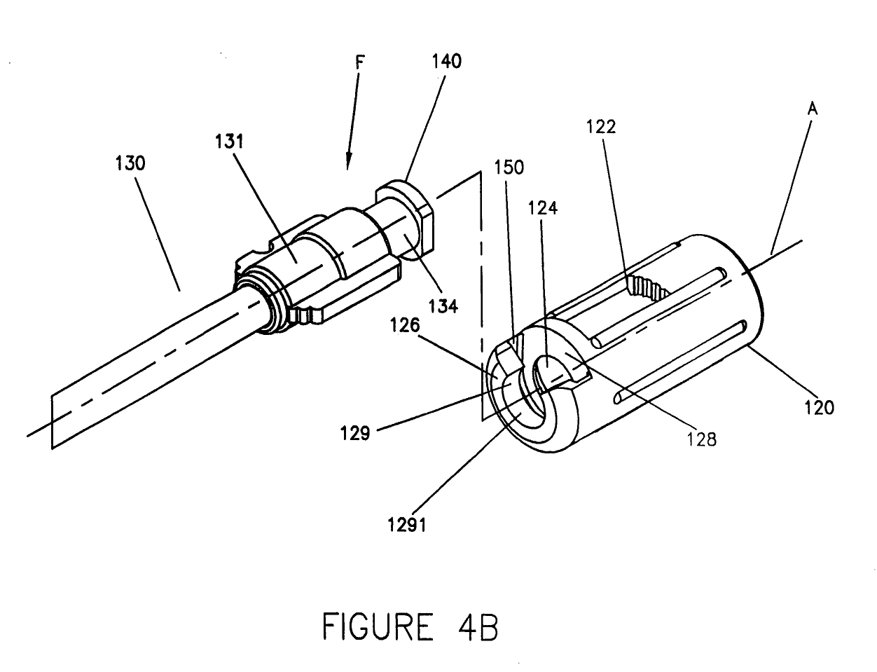

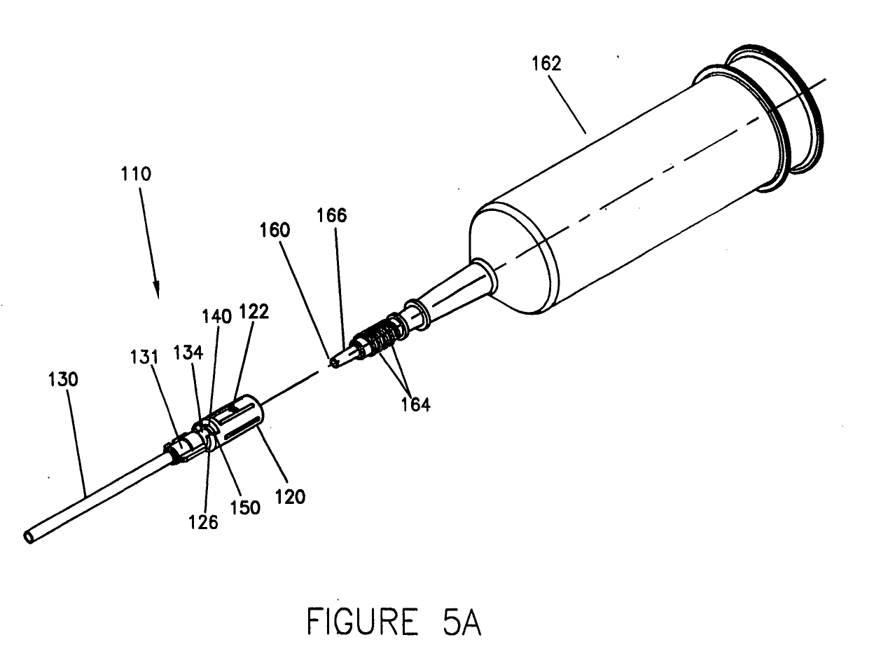

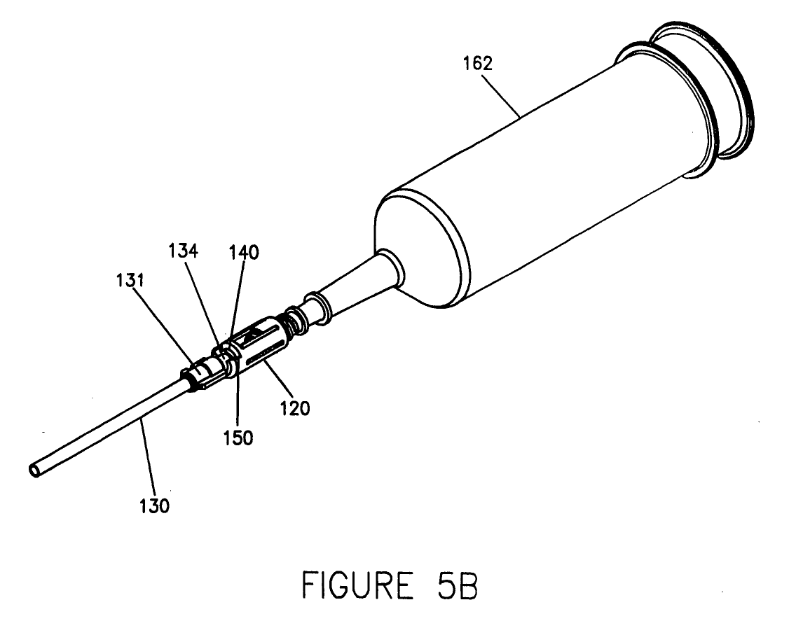

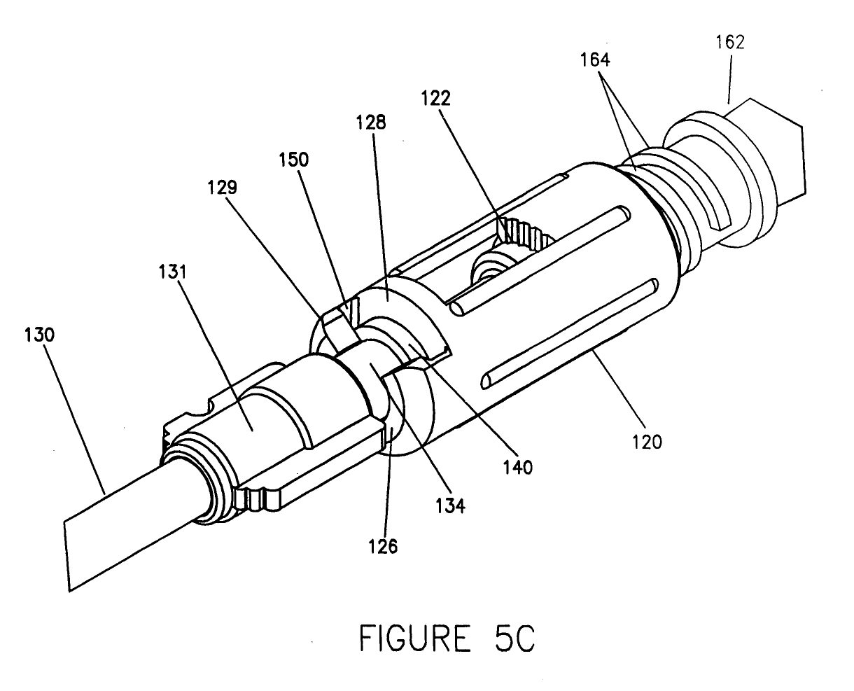

In contrast, as shown in FIG. 2, the preferred method of the present invention eliminates a number of the steps required in the conventional loading method and decreases the number of non-sterile surfaces that are handled by medical personnel: (1) a non-sterile technician in the injection suite opens the sealed tray containing a syringe; (2) the non-sterile technician installs the syringe onto the injector; (3) the non-sterile technician opens the combined syringe nut/connecting tubing assembly 110 (see FIGS. 4 and 5) package; (4) a second, sterile technician in the injection suite removes the sterile syringe nut/connecting tubing assembly 110 from the opened package; (5) the sterile technician attaches the sterile syringe nut/connecting tubing assembly 110 to the syringe; and (6) the sterile technician tightens the syringe nut on the nozzle of the syringe to make luer connection. As with the conventional method, to complete the fluid path from the syringe to the patient, the sterile technician then connects the other end of the connecting tubing to the catheter inserted in the patient to provide a sterile luer connection therebetween.

As can be appreciated, by combining the syringe nut and connecting tubing into one assembly 110, and thereby eliminating the need for a separate syringe nut package within the syringe tray, only six (6) steps are required to install the syringe and combined syringe nut/connecting tubing assembly 110 of the present invention. Consequently, by eliminating at least three (3) steps required for the conventional method and by providing less non-sterile surfaces that must be handled by medical technicians, the preferred method of the present invention saves time in preparing an injector for an injection procedure and reduces the possibility of patient contamination from non-sterile surfaces.

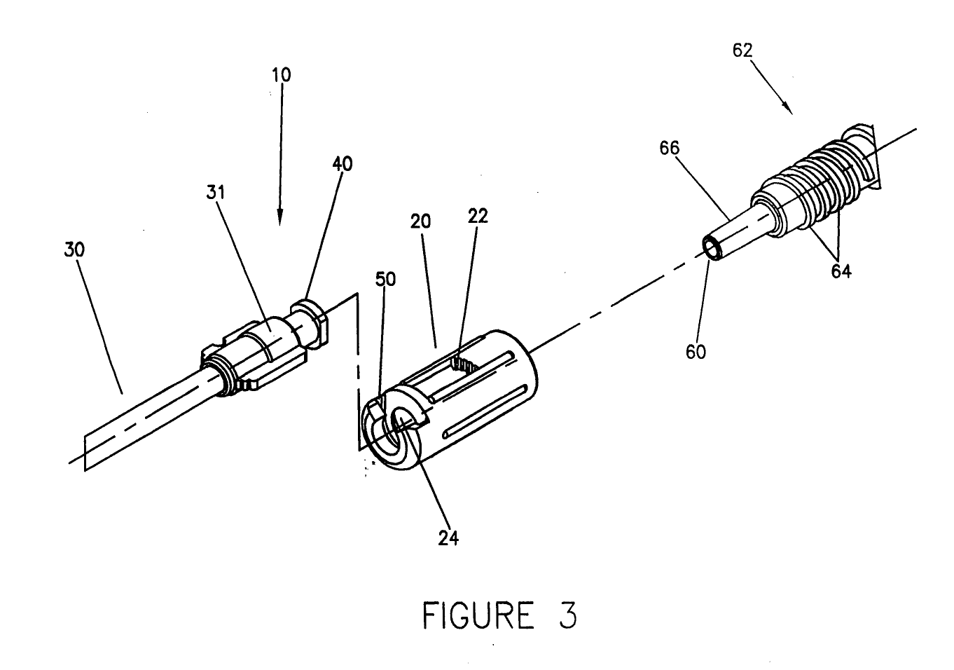

As discussed above, prior to the present invention a prefilled syringe and a separately-package syringe nut connector was provided in a first container and a length of connecting tubing was provided in a separate second container for forming a connection between the syringe and a catheter for an injection procedure. An example of such a connector/tubing combination 10 is provide in FIG. 3.

In the embodiment of FIG. 3, a connector 20 and a length of high-pressure tubing 30 may be releasably connected via cooperation of a flange 40 on a female luer connector 31 formed on or attached to the rear end of the tubing 30 and a cooperating slot 50 formed in the forward end of connector 20. Typically, a male luer connector (not shown) is formed on or attached to the front end of the tubing 30 for connection to a catheter inserted into a patient.

While maintaining connector 20 and tubing 30 connection, the operator aligns connector 20 with a tip 60 of a syringe 62 and slides connector 20 over syringe tip 60. Connector 20 typically includes a threaded portion 22 that cooperates with threading 64 on syringe 62. As connector 20 is rotated relative to syringe tip 60, a tapered end 66 of syringe tip 60 passes through an opening 24 in connector 20 to mate with a correspondingly tapered interior wall (not shown) of the female luer connector 31 on the rearward portion of tubing 30 to form a luer connection as known in the medical arts. (The length and taper angles for male and female luer connectors are governed by industry standards, such as ANSI Standard #MD-70.) After connector 20 is tightly threaded onto syringe 62, tapered end 66 and the interior wall of female luer connector 31 are forced together to create a high pressure seal, and flange 40 and slot 50 cooperate to prevent separation of connector 20 and tubing 30.

Alternatively, connector 20 can first be partly threaded onto syringe 62, taking care that tapered end 66 does not protrude through opening 24. The luer connector 31 of tubing 30 can then be attached to connector 20 by sliding flange 40 into cooperating slot 50. While taking care to maintain the alignment of the interior passage of the luer connector 31 with opening 24, connector 20 is then rotated to thread connector 20 onto syringe 62 and form a mating engagement between tapered end 66 and the interior wall of luer connector 31 of tubing 30.

As illustrated in FIGS. 4A and 4B, assembly 110 of the present invention includes a connector 120 and a length of tubing 130 that are connected in a manner to prevent disconnection thereof. Preferably, the assembly 100 is provided in a sterile package for connection to the syringe 162.

As used herein to describe assembly 110, the terms “axial” or “axially” refer generally to an axis A around which assembly 110 and syringe 162 are preferably formed 15 (although not necessarily symmetrically therearound). The terms “proximal” or “rearward” refer generally to an axial direction toward the rearward end of syringe 162 opposite a syringe tip 160. The terms “distal” or “forward” refer generally to an axial direction toward the front end (i.e., adjacent the patient catheter) of tubing 30. The term “radial” refers generally to a direction normal to axis A.

In a preferred embodiment, the syringe 162 is provided in a sterile package and the interior surface of the syringe 162 is sterile. Preferably, the syringe 162 is a prefilled syringe, which may be manufactured by and in the apparatus described in U.S. Pat. No. 5,687,542 and according to the method described in PCT International Application No. WO 97/08054, the disclosures of which are hereby incorporated by reference.

In the embodiment of FIGS. 4A and 4B, connector 120 is preferably attached to tubing 130 via a “snap fitting.” Tubing 130 includes a flange 140 at the rearward end thereof. Connector 120 includes a cooperating slot 150 into which flange 140 slides to align an inner passage of tubing 130 (not shown) with an opening 124 (see FIG. 4B) in connector 120. Flange 140 cooperates with a retaining member or flange 126 formed upon a forward end of connector 120 and a forward abutment wall 128 on connector 120 (which form slot 150 therebetween) to substantially prevent relative axial movement/separation of connector 120 and tubing 130 after connection thereof.

Retaining member 126 is preferably of a generally circular shape with an opening 129 therein. Opening 129 allows passage of a generally cylindrical portion 134 of tubing 130 therethrough when connector 120 and tubing 130 are connected. The width of opening 129 is preferably somewhat smaller than the diameter of generally cylindrical portion 134, such that the connector 131 of tubing 130 snaps into place when aligned with connector 120 and sufficient force is applied in the direction of arrow F (preferably during fabrication of assembly 110). To facilitate such a snap fitting, connector 120 is preferably fabricated from a resilient polymeric material, such as polycarbonate (PC). In addition, the connector 131 is preferably fabricated from rigid polyvinylchloride (PVC) and the tubing 130 is preferably fabricated from flexible PVC.

Because opening 129 is smaller than cylindrical portion 134 of tubing 130, retaining member 126 prevents disconnection of connector 120 and the connector 131 of tubing 130 after fabrication thereof. As used herein, the phrase “prevents disconnection” does not mean that connector 120 and connector 131 are impossible to disconnect. In that regard, it may be possible to apply sufficient force on connector 131 of tubing 130 to cause opening 129 to enlarge sufficiently to allow disconnection of connector 120 and tubing 130.

Preferably, however, connector 120 and tubing 130 of assembly 110 will remain in a connected state under all circumstances and forces normally experienced before and during connection of assembly 110 to syringe 162. Assembly 110 can, for example, be designed to prevent disconnection of connector 120 and tubing 130 without breaking one of connector 120 and connector 131 by appropriate choice of materials and/or appropriately sizing opening 129.

As an alternative to a “snap fit” design as described above, an assembly of the present invention can be formed, for example, via a technique know as In-Mold Assembly in which parts can be molded as an assembled product in an injection molding cycle. See, for example, “Manufacturing”, Injection Molding, pp. 81-83 (February 1998).

In-Mold Assembly, which was developed by Fickenscher America, L.L.C., is a multi-shot injection molding process whereby the first shot of material produces a preform that is used as the mold cavity geometry for subsequent molding shots. Because each shot comprises a different material having different melt temperatures, no bonding occurs between the parts being molded.

According to a preferred method of manufacturing, the preform (first shot) is injection molded and allowed to cool (solidify). The preform is then transferred to a new insert, either by hand or mechanically, to be used as the cavity geometry for the second shot. The plastic material for the second shot has a melt temperature of at least 50° F. less than the first material. This insures that the first material will not begin to melt when the second material comes into contact with it. Because the first material does not melt during the second shot, as the second shot cools it shrinks away from the preform and creates a small gap between the two parts allowing for free rotation.

In an In-Mold Assembly procedure, the connector and the tubing (or at least the rearward portion of tubing 130) of the present invention can be injection molded as an assembled product. Such a product would look very similar to assembly 110 of FIGS. 4A and 4B. Because the connector 120 and tubing 130 would be molded together, the opening 129 in retention member 126 would not be required and there would be no need for the “snap fit” connection during manufacture of the assembly 110. Further, because no bonding takes place between the molded materials, the luer fitting 131 will be free to rotate within the connector 120, thereby forming a finished part that does not require any secondary operation to mechanically assemble the connector 120 and luer fitting 131.

Regardless of the manufacturing techniques used to fabricate assembly 110, connector 120 and connector 131 of tubing 130 are preferably rotatable around axis A relative to each other to facilitate connection of assembly 110 to syringe 162. In the embodiment of FIGS. 4A and 4B, flange 140 is preferably easily rotatable around axis A within slot 150 and cylindrical portion 134 is preferably easily rotatable within a generally circular passage 1291 formed (in communicative connection with opening 129) in connector 120.

The connection of assembly 110 to syringe 162 is illustrated in FIGS. 5A through 5C. Assembly 110 is preferably first aligned with syringe tip 160 as illustrated in FIG. 5A. Connector 120 of assembly 110 is then slid over syringe tip 160. Connector 120 preferably includes a threaded portion 122 that cooperates with a threaded portion 164 on syringe 162. As connector 120 is rotated relative to syringe tip 160, tapered end 166 of syringe tip 160 passes through opening 124 in connector 120 to mate with a correspondingly tapered interior wall (not shown) on the rearward portion of tubing 130 to form a luer connection as known in the medical arts.

Assembly 110 of the present invention assists in maintaining proper alignment of connector 120, tubing 130 and syringe 162 during connection thereof. To further facilitate proper alignment, threaded portion 122 of connector 120 is preferably positioned such that tapered portion 166 of syringe tip 160 passes through opening 124 and into the interior of tubing connector 131 before threading portion 122 engages threading 164. This relative positioning of threaded portion 122 and threading 164 of syringe 162 also reduces the amount of rotation of connector 120 relative to syringe 162 required to form a sealed engagement as compared to currently available connector/tubing combinations.

The connection of assembly 110 of the present invention to syringe 162 is in many respects similar to the connection of currently available connector/tubing combinations (for example, as illustrated in FIG. 1). However, while operators of injection procedures will appreciate the familiar method of attaching assembly 110 to syringe 162, assembly 110 provides a number of significant advantages over currently available connector/tubing combinations. In that regard, because connector 120 and tubing 130 of assembly 110 are prevented from disconnecting, it is much easier and quicker for the operator to connect assembly 110 to syringe 162 without the alignment problems typically associated with currently available connector/tubing combinations. Moreover, sterile assembly 110 is preferably shipped or transported in a single sterile package, potentially reducing packaging costs as compared to currently available connector/tubing combinations. Furthermore, because connector 120 and tubing 130 of assembly 110 are prevented from disconnecting, the risk of contamination of the interior wall of tubing 130 during handling by the operator is greatly reduced.

The present invention provides a connector and tubing assembly 110 that is pre-connected and sterile, thereby reducing assembly steps and sterility concerns for injection procedures. Furthermore, because a separate syringe nut connector package is not required for the present invention, packaging costs are reduced. Moreover, in the preferred embodiment, because the interior surface of the syringe 162 is sterile and the assembly 110 is sterile, a sterile operator may quickly and conveniently connect the syringe 162 and the assembly 110 together to provide a sterile pathway from the syringe to the patient.

It should be appreciated that the present invention, including the assembly 110, may be configured as appropriate for the application. The embodiments described above are to be considered in all respects only as illustrative and not restrictive. The scope of the invention is indicated by the following claims rather than by the foregoing description. All changes which fall within the meaning and range of equivalency of the claims are to be embraced within their scope.

1. A syringe assembly comprising:
a syringe comprising a distal end terminating in a tip, and an externally-threaded portion;
a tubing comprising an end portion with an end flange; and
a connector defining a bore with an internally-threaded portion for cooperating with the externally-threaded portion of the syringe, and comprising a retaining member forming an end of the connector and defining a first opening, and an abutment wall spaced from the retaining member and defining a second opening connecting to the bore, the retaining member and abutment wall defining a slot therebetween for receiving the end flange;
wherein the bore is adapted to receive the syringe distal end such that the syringe tip passes through the second opening before the internally-threaded portion of the bore engages the externally-threaded portion of the syringe.

2. The syringe assembly of claim 1 wherein the slot has a width smaller than the width of the end flange, such that an interference engagement is formed between the end flange and connector when the end flange is received in the slot.

3. The syringe assembly of claim 2 wherein the interference engagement substantially prevents relative axial movement between the connector and tubing.

4. The syringe assembly of claim 2 wherein the first opening is generally C-shaped and the tubing end portion is generally cylindrical-shaped to engage the C-shaped first opening when the end flange is received in the slot.

5. The syringe assembly of claim 1 wherein the tubing end portion defines a female luer and the syringe distal end is tapered for engaging the female luer when the syringe tip is inserted through the second opening.

6. The syringe assembly of claim 1 wherein the slot is adapted to rotatably receive the end flange.

7. The syringe assembly of claim 1 wherein the syringe comprises a pre-filled syringe.

8. The syringe assembly of claim 1 wherein the connector is formed of a resilient polymeric material.

9. The syringe assembly of claim 1 wherein the connector and tubing end portion are formed of a resilient polymeric material.

10. A method of associating tubing with a syringe comprising:
providing a syringe comprising a distal end terminating in a tip and an externally-threaded portion, a tubing comprising an end portion with an end flange, and a connector defining a bore with an internally-threaded portion and comprising a retaining member forming an end of the connector and defining a first opening and an abutment wall spaced from the retaining member and defining a second opening connecting to the bore, the retaining member and abutment wall defining a slot therebetween; and
inserting the syringe distal end into the bore such that the syringe tip passes through the second opening before the internally-threaded portion of the bore engages the externally-threaded portion of the syringe.

11. The method of claim 10 further comprising inserting the end flange into the slot.

12. The method of claim 11 wherein the end flange is rotatably received in the slot.

13. The method of claim 10 further comprising threading the externally-threaded portion of the syringe into engagement with the internally-threaded portion of the bore to secure the syringe to the connector.

14. The method of claim 10 wherein the tubing end portion defines a female luer and the syringe distal end is tapered, such that the step of inserting the syringe distal end into the bore comprises the syringe distal end engaging the female luer as the syringe tip passes through the second opening.

15. A method of manufacturing a connector and tubing assembly comprising:
molding a connector defining a bore from a first polymeric material having a first melting point;
cooling the connector;
molding an end portion adapted for connection to a tubing at least partially within the connector bore from a second polymeric material having a second melting point that is less than the first melting point; and
cooling the end portion, such that the portion of the end portion molded within the connector bore shrinks from contact with the connector such that the end portion is rotatable relative to the connector.

16. The method of claim 15 wherein the end portion is molded with a female luer connection.

17. The method of claim 15 wherein the connector is molded with an internally-threaded portion within the connector bore.

18. The method of claim 15 wherein the connector is molded as a generally cylindrical body defining the central bore.

19. The method of claim 15 wherein the portion of the end portion molded within the connector bore is molded with an interfering end flange.

20. The method of claim 15 wherein the second melting point is at least 50° F. less than the first melting point.
